(19) United States
(12) Patent Application Publication
(10) Pub. No.: US 2008/0262295 A1
(43) Pub. Date: Oct. 23, 2008

(54) METHODS AND DEVICES FOR VIEWING ANATOMIC STRUCTURE

(76) Inventors: Amar Kendale, Newton, MA (US); John Tamkin, San Marino, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/053,029

(22) Filed: Mar. 21, 2008

Related U.S. Application Data
(60) Provisional application No. 60/896,394, filed on Mar. 22, 2007.

(51) Int. Cl. A61B 1/005

(52) U.S. Cl. 600/104; 600/175; 600/153

(57) ABSTRACT

Disclosed herein are various systems and methods for improving the visualization of an optical device, in particular a medical device. The device can include a lens cover that provides an offset between the distal end of an endoscope and the surrounding environment. At least a portion of the lens cover can have an ellipsoidal shape.

This application claims priority to Provisional Application Ser. No. 60/896,394, entitled “Methods And Devices For Viewing Anatomic Structure” filed Mar. 22, 2007, which is incorporated herein by reference

### BACKGROUND OF THE INVENTION

The ability to view internal anatomy has lead to significant advances in surgical capability and diagnosis. In particular, the number of procedures that can be performed in a minimally-invasive manner has increased, while such procedures have, in general, improved patient outcomes and reduced recovery time.

One such optical device is a conventional endoscope. Endoscopes generally include an elongate body that can be maneuvered to a target site through a body cavity and/or through a small incision. Observation of tissue inside the body can then be carried out with minimal patient trauma. Similarly, endoscopes can permit visualization of a surgical site so that a clinician can perform various medical procedures.

In order to allow visualization, the distal end of endoscopes include some type of image collection element (e.g., a lens or a sensor). An image is received by the image collection element and then transmitted through the flexible body. A viewer associated with the proximal portion of the endoscope allows a clinician to view anatomy adjacent to the image collection element.

However, conventional endoscopes can collect bodily substances on the image collection element when the distal end of the device contacts bodily fluids and/or tissue. Smearing of the image collection element and blurring of the image can require removal and cleaning of the endoscope. In addition, when the distal end of the endoscope is placed against tissue, visualization is limited to the tissue surface. In order to overcome these drawbacks, insufflation fluid can be delivered to expand a body cavity and increase the visualization area. Alternatively, the clinician can carefully maneuver the endoscope in an attempt to avoid smearing and/or to view a desired tissue structure.

Accordingly, further improvements to optical devices, particularly improvements that facilitate visualization, would be beneficial.

### SUMMARY OF THE INVENTION

Disclosed herein are methods and devices for viewing anatomic structure with improved visualization. In one embodiment, a lens cover is disclosed for providing an offset between an optical device and the adjacent environment. The lens cover can be positioned proximate to the image collection element of a medical device and extend distally therefrom. The distal end of the lens cover can be configured to allow visualization through a transparent distal portion that includes a prolate spheroidal shape.

In another aspect, the prolate spheroidal shape is an ellipsoid. For example, the distal end of the lens cover can have an inner and outer surface with a generally ellipsoid shape.

In another aspect, the lens cover further comprises a proximal mating section. The mating section can be configured for mating with a distal portion of a medical device. In one aspect, the medical device is an endoscope. In another aspect the medical device comprises a cannula for receiving an endoscope. In yet another aspect, at least a portion of the endoscope is formed integrally with the medical device.

In another embodiment, the lens cover includes a first and second distal section. The first distal section has a prolate spheroidal shape and the second distal section has a shape different from the first section. In addition, the lens cover can have a proximal mating section for mating with a medical device.

In another aspect, the prolate spheroid tip is aligned with a viewing field of an endoscope such that endoscopic images are viewed through the prolate spheroid tip. In addition, a light emitter can be configured for directing light through at least a portion of the lens cover.

The offset provided by the lens cover can be defined by the portion of the lens cover having a prolate spheroidal shape. In another aspect, the offset is defined by a first and second section where the inner and outer surfaces of the first section have a prolate spheroidal shape and the inner and outer surface of the second section have a different shape. In one aspect, the inner and outer surfaces of the second section have a cylindrical shape.

In another aspect, the lens cover provides an offset between an endoscope and the distal end of the lens cover of at least about 11 mm. In another aspect, the lens cover is adapted to provide an offset in the range of about 12 mm and 15 mm. In yet another aspect, the lens cover is adapted to provide an offset in the range of about 13 mm and 14 mm.

### BRIEF DESCRIPTION OF THE DRAWINGS

The accompanying drawings, which are incorporated in and constitute a part of this specification, illustrate exemplary embodiments of the invention and, together with the description, serve to explain the principles of the invention.

FIG. 1 is a side view of one exemplary embodiment of a medical device for use with the lens cover described herein;

FIG. 2 is a cross-sectional view of one exemplary embodiment of the lens cover described herein;

FIG. 3 is a cross-sectional view of another exemplary embodiment of the lens cover described herein; and

FIG. 4 is a cross-sectional view of yet another exemplary embodiment of the lens cover described herein.

### DETAILED DESCRIPTION

Disclosed herein are methods and devices for viewing anatomic structure, particularly devices and system that allow improved visualization. In one such embodiment, a lens cover provides an offset between an optical device, such as an endoscope, and adjacent tissue. The lens cover can directly mate with an endoscope and/or mate with a medical instrument adapted to receive an endoscope. Generally, the distal end of the lens cover can have a configuration that allows visualization therethrough. For example, the lens cover can have a transparent distal portion including a prolate spheroid tip. In another aspect, the lens cover can have an ellipsoid shape. The offset provided by the lens cover can improve visualization when the medical instrument is in contact with tissue. At the same time, the lens shape provides an acceptable trade-off in image distortion, atraumatic design, and multi-medium visualization capability.

While the term “endoscope” is used herein, one skilled in the art will appreciate that a variety of optical devices can work with the disclosed lens cover. In particular, the term “endoscope” is intended to include the variety of optical devices that allows a user to view an image at a distance through an elongate body.

When a clinician directs a conventional endoscope through a patient, a variety of mediums are encountered and the different properties of those mediums can present challenges to visualization equipment. When an endoscope lens contacts tissue, the image provided by the endoscope is limited to the immediate tissue surface. And, in addition, contact with tissue can transfer biomaterial that smears the endoscope detector and blurs images. If image quality degrades sufficiently, the clinician has to remove the endoscope, clean the detector, and reinsert the endoscope. On reinsertion, the lens can again become obscured and require repeated removal and cleaning.

Conversely, the lens cover described herein can reduce the difficulties with tissue contact by providing an offset between the distal end of the endoscope and the environment. The offset can allow visualization when the distal tip of the lens cover is in contact with tissue and/or when the distal tip of the lens cover becomes smeared. In one aspect, the lens cover can have an atraumatic shape for use in delicate procedures, while allowing for sufficient offset and viewing in a variety of environments.

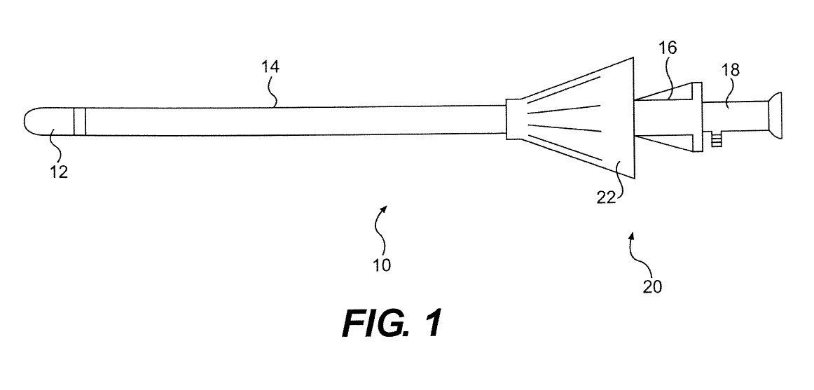

FIG. 1 illustrates one exemplary embodiment of a device 10 that can be used with the lens cover described herein. The device includes an elongate body 14 for traversing a body lumen and/or surgical pathway, with lens cover 12 positioned at the distal end thereof. While lens cover 12 is illustrated at the distal-most end of device 10, the lens cover can alternatively be positioned on a more proximal portion of body 14. Body 14 can have a flexible or rigid configuration and can house an optical device and/or have at least one channel for receipt of an optical device.

In particular, body 14 can define an endoscope body or a cannula of a device for use with an endoscope. Where the body of device 10 provides a cannula for working with an endoscope, a proximal end 20 of device 10 can include at least one port or opening for receipt of an optical device, such as, an endoscope. For example, a endoscope port 16 can receive endoscope 18. Alternatively, an endoscope, or a portion of an endoscope, can be integrated into device 10. For example, a camera can be integrated into the proximal end of the device and connected to a viewing screen when the device is in use.

In addition, device 10 can include a light emitter. In one aspect, the light emitter is positioned adjacent to lens cover 12 and configured to direct light through lens cover 12. In another aspect, described in more detail below, the endoscope includes a light emitter.

Depending on the intended use of device 10, additional instruments can be directed through additional openings in the proximal end of device 10. Proximal end 20 can also include a handle 22 that allows a user to grasp and manipulate device 10. Alternatively, or additionally, handle 22 can be configured for mating with a frame to hold the device in place during use.

Lens cover 12 can allow a user to view the environment outside of device 10 via an image collection element located within device 10. When a user inserts an endoscope into device 10, the distal end of the endoscope can be positioned proximate to the lens cover. In one aspect, device 10 includes a stop to limit distal movement of the endoscope once the distal end of the endoscope is positioned relative to the lens cover. For example, a user can move the endoscope through a channel in body 14 until distal movement is prevented by a stop. In one aspect, the stop is located in the distal portion of the device. For example, the lens cover 12 or a portion of body 14 proximate to the lens cover can include an area of reduced diameter to limit the distal movement of the endoscope. In another aspect, the stop is located proximally. For example, endoscope port 16 can limit distal movement of the endoscope once the endoscope is properly situated.

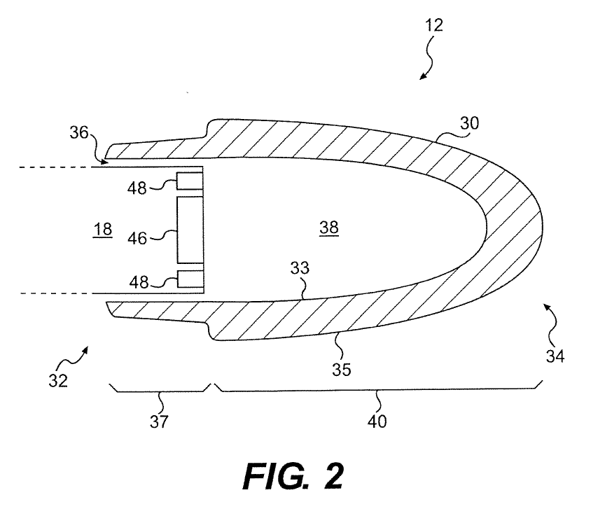

FIG. 2 illustrates one exemplary embodiment of lens cover 12 having a generally elongate body 30 extending between a proximal end 32 and a distal end 34 and including an inner surface 33 and outer surface 35. Proximal end 32 can include an opening 36 for receipt of an endoscope into an open interior region 38. In addition, a proximal portion 37 of the lens cover can be configured for mating with a medical device, such as, device 10. Where the device mates with the surface of the lens cover, the proximal portion can include mating features. For example, the inner and/or outer surfaces of lens cover body 30 can mate with device 10 via a mechanical, frictional, and/or adhesive connection.

The distal end of lens cover body 30 can have a size and shape for moving through a body passage while causing minimal trauma. Generally, a distal portion 40 of the lens cover has a blunt distal tip provided by a curved outer surface.

Lens cover body 30 can be formed of a variety of biocompatible materials, particularly transparent biocompatible materials such as polymers, elastomers, and glass. However, the entire body need not be formed of a transparent material. For example, in one aspect, the proximal portion 37 of lens cover body 30 does not require the transmission of light. In particular, where proximal portion 37 mates with device 10, the proximal portion need not be formed of a transparent material.

In one aspect, as illustrated in FIG. 2, body 30 is configured for the receipt of the distal end of endoscope 18 via proximal opening 36. Endoscope 18 can include both a visualization portion 46 and a light emitting portion 48. The light emitting portion 48 can transmit light through the lens cover to illuminate at least a portion of the environment adjacent to the lens cover. Similarly, the visualization portion 46 of endoscope 18 can allow a user to view the adjacent environment through the lens cover. The light emitting portion 48 can represent the variety of light emitters used with conventional endoscopes, and in one aspect, the light emitting portion 48 is a light ring.

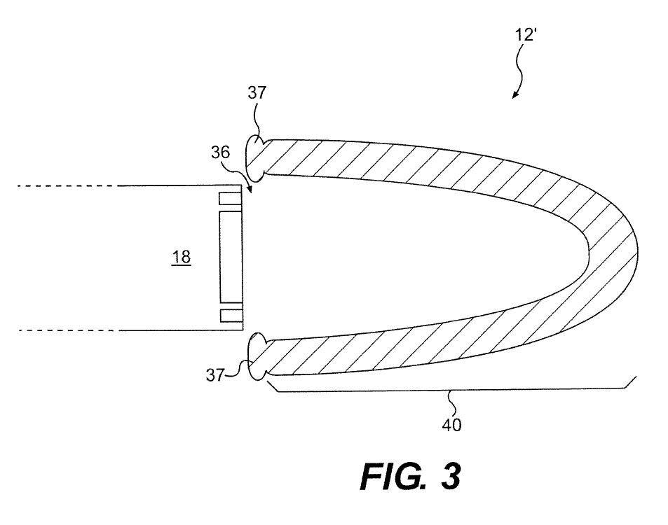

In another embodiment, the endoscope need not extend into the interior portion of the lens cover. For example, FIG. 3 illustrates lens cover 12′ with endoscope 18 positioned on the proximal side of opening 36. Mating portion 37 is sized such that the distal end of endoscope 18, while positioned outside of lens cover 12′, can view an image through the lens cover. In one aspect, mating portion 37 has a small length or is defined by the proximal surface of distal portion 40. Lens cover 12′ comprises a distal portion 40 similar to lens cover 12 described above.

Distal portion 40 of the lens covers 12, 12′ described above are configured to provide an offset between the distal end of the endoscope and the distal tip of the lens cover. When the lens cover contacts tissue, the offset allows a user to visualize more than the tissue immediately adjacent to the distal end of the lens cover. In particular, the user can visualize a greater tissue area and/or adjacent fluid environment. This increased visualization area can facilitate movement of a medical device, such as device 10, along a pathway through a patient.

Obtaining sufficient offset while minimizing image distortion, however, is a challenge. Applicants realized that achieving a sufficient offset could best be attained with a lens cover where a portion of the lens cover has a prolate spheroidal shape. In particular, the distal-most portion of the lens cover can be defined by a portion of a prolate spheroid. While a spherical shape can reduce image distortion, insufficient offset is provided. Similarly, while a conical shape can provide sufficient offset, the cone results in increased image distortion. In particular, a conical lens limits the view of the endoscope directly in front (i.e., 0°) of the lens cover and has a limited viewing angle. While a liquid environment will increase the viewing angle, the conical lens is not as effective in gaseous environment. Conversely, a prolate spheroidal shape can provide an adequate viewing angle in both a liquid and gaseous environment. Moreover, where the outer surface of the lens cover has a conical shape, the lens cover converges to a point. In certain applications where the lens cover is used to view sensitive tissue, the use of a blunt distal surface can be desirable.

In one aspect, the prolate spheroidal shape is defined by a portion of an ellipsoid. The ellipsoid shaped portion of the lens cover can extend from the distal most part of the lens cover. In one aspect, distal portion 40 of lens cover 12 is defined by a half ellipsoid, cut across its width (e.g., cut perpendicular to the major axis of the ellipsoid). In another aspect, the distal portion 40 is defined by less than half of an ellipsoid. For example, the distal most portion of the lens cover can have a curvature which approximates a shaped defined by about 10% to about 90% of an ellipsoid, in another aspect the size of the shape is defined by about 10% to about 50% of an ellipsoid, and in yet another aspect the shape is defined by about 15% to about 35% of an ellipsoid. The percentage can be measured based on the area of the ellipse or as the distance along the major axis from the outside of the ellipsoid. The size of the ellipsoid can be chosen based on the desired offset between the endoscope and the distal tip of the lens cover, the viewing angle of the endoscope camera, the location of the endoscope camera, the location of a light source, and/or the intended use of the device.

In another aspect, the size of the ellipsoid can be chosen such that the location of an ellipse foci falls on the image collection element. Placing one of the foci on the image collection element can provide an atraumatic lens cover tip.

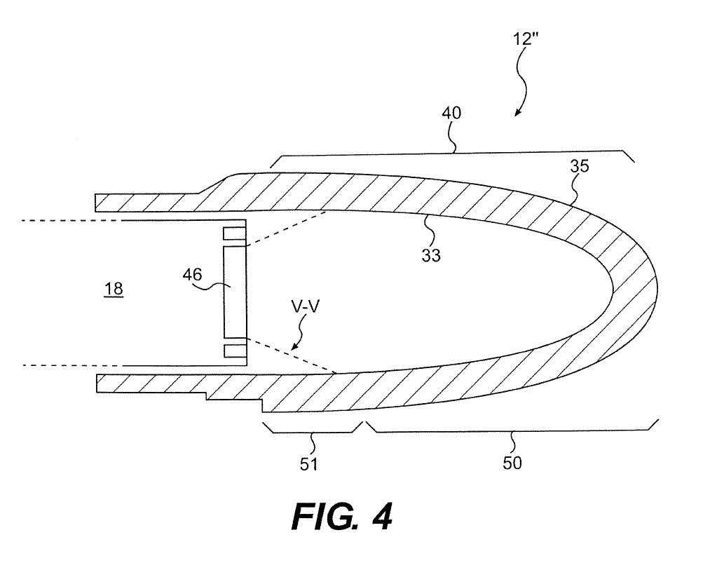

In another embodiment, the distal portion 40 of lens cover 12, 12′ can include a first section having a prolate spheroidal shape and a second section having a different shape. FIG. 4 illustrates an exemplary embodiment of lens cover 12″ having a first section 50 and a second section 51. In one embodiment, visualization is achieved through the portion of the lens cover defined by the prolate spheroid. Depending on the endoscope used with the lens cover 12″, visualization portion 46 of endoscope 18 has less than a 180° field of view. The lens cover can be positioned such that the field of view of the endoscope generally matches up with the first section 50 of lens cover 12″. As a result, the endoscope views the environment around the lens cover through the portion of the lens cover defined by a prolate spheroid.

The second section 51 of the lens cover can allow light to pass therethrough, but does not require the optical properties provided by the prolate spheroid first section. As a result, second section 51 can have a variety of shapes. For example, the cross-sectional shape of the second section 51 can be circular, rectangular, triangular, oval, and/or irregular. In addition, the cross-sectional shape of the second section 51 can vary along the length of the lens cover to provide a tapered or conic segment.

As mentioned above, the lens cover can be sized and shaped such that the endoscope views the surrounding environment substantially though the portion of the lens cover having a prolate spheroidal shape. In one aspect, second section 51 is sized such that the first section begins at the point at which an imaginary line V-V, defined by the viewing angle of the endoscope, intersects the lens cover. Thus, in one embodiment, the length of the second section 51 depends on the viewing angle of the endoscope and the location of the endoscope relative to the lens cover. However, depending on the intended use of device 10, the imaging capabilities of the endoscope, and the desired shape and size of the lens cover, second section 51 could have a variety of different lengths.

With respect to the “shape” of the lens cover discussed above, the inner surface 33 of the lens cover 12, 12′, 12″ can have a similar configuration to the outer surface 35. In one embodiment, the shapes of inner surface 33 and outer surface 35 are defined by a portion of a prolate spheroid. In another aspect, both the inner and outer surfaces shapes are defined by a portion of an ellipse. In general, the inner and outer surface can have corresponding shapes such that the inner surface shape matches the outer surface shape, but has different dimensions.

In another embodiment, the inner and outer surface 33, 35 can have different shapes. For example, the inner surface or outer surface could be defined by an ellipse, while the other surface has a prolate spheroidal shape.

The inner and outer surfaces 33, 35 need not exactly match a prolate spheroidal shape. For example, the distal-most surface can have a flattened end or a surface feature to facilitate tissue grip. For example, the distal-most outer surface can have a slight recess to allow the lens cover to grip tissue and move tissue or hold tissue. In another aspect, the inner surface can vary from the prolate spheroidal shape at its distal-most end. As described in a co-pending application entitled “Methods and Devices for Reducing Reflection-induced Artifacts” filed on even day herewith, and incorporated by reference in its entirety, the inner surface can mate with a blocking element to reduce the occurrence of reflection induced image artifacts. The connection between the lens cover and the blocking element may require a small variation in the shape of the inner and/or outer surface of the lens cover.

In another example of a departure from the prolate spheroidal shape, the lens cover can have a hybrid ellipsoidal shape, such as, for example a hybrid conical/elliptical shape. As mentioned above, a conical lens does not provide the desired optical and atraumatic properties. However, where some dissection with the lens cover is contemplated, a hybrid conical-ellipsoidal shape can be used. The hybrid lens balances intentional dissection capability with limited image distortion.

As mentioned above, the lens cover provides an offset between the distal end of the endoscope and the distal end of the lens cover. The amount of offset can vary depending on the width of the lens cover and/or the intended use of the device associated with the lens cover. In one aspect, where the lens cover is used for cardiac applications, such as, for example viewing and/or maneuvering around epicardial tissue, the offset provided by the lens cover can be at least about 11 mm. In another aspect, the offset can be in the range of about 8 to 25 mm, in yet another aspect in the range of about 10 to 20 mm, and in still yet another aspect in the range of about 12 to 15 mm. In still another embodiment, the offset is about 13 to 14 mm. The chosen offset distance can depend on the width of the lens cover, particularly where at least a portion of the lens cover has an elliptical shape. In applications that allow for wider lens covers, the offset distance can be increased. Similarly, where the width of the lens cover is more limited, a shorter offset distance can be chosen.

As mentioned above, the offset represents the distance between the distal end of the endoscope and the distal end of the lens cover. Where optical components are incorporated into device 10 the offset can be measured between the distal surface of the optical components and the distal end of the lens cover. The examples described below illustrate two lens covers. In Example One, the distal end of the lens cover has an ellipsoidal inner and outer surface shape.

### EXAMPLE ONE

Exemplary Lens Cover One

| Total Length | 14 | mm |
|---|---|---|
| External Surface | | |
| radius | 1.1668 | mm |
| conic | −0.88895 | |
| semi-minor axis | 3.5 | mm |
| maximum external | 7.0 | mm |
| diameter | | |
| Internal Surface | | |
| radius | 0.63888 | mm |
| conic | −0.93275 | |
| semi-minor axis | 2.46 | mm |
| maximum external | 4.92 | mm |
| diameter | | |
| vertex separation | 1.61 | mm |

In Example Two, the distal end of the lens cover has a distal surface with a first section defined by a cylinder and a second section defined by an ellipsoid inner and outer surface.

### EXAMPLE TWO

Exemplary Lens Cover Two

| Total Length | 20 | mm |
|---|---|---|
| External Surface | 4.5 | mm |
| Length of | | |
| Cylindrically | | |
| Shaped Section | | |
| External Ellipsoid | | |
| Surface | | |
| radius | 2.32258 | mm |
| conic | −0.85016 | |
| semi-minor axis | 6.0 | mm |
| maximum external | 12.0 | mm |
| diameter | | |
| Internal Ellipsoid | | |
| Surface | | |
| radius | 1.27485 | mm |
| conic | −0.92412 | |
| maximum external | 9.26 | mm |
| diameter | | |
| vertex separation | 1.61 | mm |

| | |
|---|---|
| field of view in air | +/−28° |
| field of view in water | +/−50° |

As illustrated in Example Two, the use of an ellipsoidal shaped inner and outer surface allows an expansive field of view in both a gaseous and liquid environment. In addition, Applicants found that the lens covers of both Examples One and Two had an acceptable amount of light reflect off the inner and outer surfaces of the lens covers from a light source that emitted light though the lens cover.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A medical device, comprising:
an elongate body having a lumen extending from a proximal opening to a distal end, where the lumen is sized and shaped for receipt of an optical device;
a stop configured to limit distal movement of an optical device relative to the elongate body without preventing visualization;
a lens cover positioned proximate to the distal opening, and extending distally from the lumen, the lens cover providing an offset, when an optical device is placed within the lumen, between the distal end of an optical device and the distal end of the lens cover, the distal end of the lens cover configured to allow visualization therethrough, wherein the lens cover has a transparent distal portion including a prolate spheroidal shaped tip.

2. The medical device of claim 1, further comprising an endoscope positioned within the lumen, the endoscope having a distal end.

3. The medical device of claim 2, wherein the prolate spheroid tip is aligned with the viewing field of the endoscope such that endoscopic images are viewed through the prolate spheroid tip.

4. The medical device of claim 1, wherein the prolate spheroid is an ellipsoid.

5. The medical device of claim 1, wherein the lens cover is adapted to provide an offset of at least about 11 mm.

6. The medical device of claim 1, wherein the lens cover is adapted to provide an offset in the range of about 12 mm and 15 mm.

7. The medical device of claim 1, wherein the lens cover is adapted to provide an offset in the range of about 13 mm and 14 mm.

8. The medical device of claim 1, wherein the offset provided by the lens cover is defined by the portion of the lens having a prolate spheroidal shape.

9. The medical device of claim 1, wherein the offset provided by the lens cover is defined by a first portion of the lens cover having a prolate spheroidal shape and a second portion of the lens cover having a different shape.

10. The medical device of claim 9, wherein the second portion has a cylindrical shape.

11. The medical device of claim 1, wherein the elongate body includes at least one additional lumen.

12. The medical device of claim 11, wherein the at least one additional lumen has an open distal end for delivery of a surgical tool.

13. The medical device of claim 1, wherein the stop is positioned proximate to the distal end of the elongate body.

14. The medical device of claim 13, wherein the stop is defined by a portion of the lens cover.

15. The medical device of claim 14, wherein the offset is measured between the stop and the distal end of the lens cover.

16. The medical device of claim 1, wherein the entire lens cover is formed of a transparent material.

17. A medical device lens cover, the device comprising:
an elongate body having an image collection element; and
a lens cover positioned proximate to the image collection element, and extending distally thereform, the lens cover providing an offset between the image collection element and the distal end of the lens cover, the distal end of the lens cover configured to allow visualization therethrough, wherein the lens cover has a transparent distal portion including a prolate spheroid tip.

18. The medical device of claim 17, wherein the image collection element is defined by the distal end of an endoscope.

19. The medical device of claim 17, wherein the prolate spheroid tip is aligned with the viewing field of the image collection element such that images are viewed through the prolate spheroid tip.

20. The medical device of claim 17, wherein the prolate spheroid is an ellipsoid.

21. The medical device of claim 17, wherein the lens cover is adapted to provide an offset of at least about 11 mm.

22. The medical device of claim 17, wherein the lens cover is adapted to provide an offset in the range of about 12 mm and 15 mm.

23. The medical device of claim 17, wherein the lens cover is adapted to provide an offset in the range of about 11 mm and 14 mm.

24. The medical device of claim 17, wherein the offset provided by the lens cover is defined by the portion of the lens cover having a prolate spheroidal shape.

25. The medical device of claim 17, wherein the offset provided by the lens is defined by a first portion of the lens having a prolate spheroidal shape and a second portion of the lens having a different shape.

26. A lens cover for use with a medical device, the device comprising:
a lens cover having an elongate body extending between a proximal mating section for mating with a distal portion of a medical device and a distal optical section, the distal optical section having a closed distal end, an inner surface, and an outer surface, at least a portion of the inner surface and the outer surface having a prolate spheroidal shape, wherein the distal optical section defines an offset of at least about 11 mm.

27. The lens cover of claim 26, wherein the prolate spheroidal shape is an ellipsoidal shape.

28. The lens cover of claim 26, wherein the elongate body further comprises an intermediate section having a shape different from the distal optical section.

29. The lens cover of claim 28, wherein the intermediate section has a cylindrical shape.

30. The lens cover of claim 26, wherein the proximal mating section includes an open proximal end.

31. The lens cover of claim 26, wherein the offset is in the range of about 12 and 15 mm.

32. The lens cover of claim 26, wherein the offset is in the range of about 13 and 14 mm.
